(19) United States
(12) Patent Application Publication
(10) Pub. No.: US 2023/0113501 A1
(43) Pub. Date: Apr. 13, 2023

(54) TREATMENT OF LEUKEMIAS AND LYMPHOMAS WITH COMBINATIONS OF BCL-2 INHIBITORS AND PLK1 INHIBITORS

(71) Applicant: Cardiff Oncology, Inc., San Diego, CA (US)

(72) Inventors: Maya RIDINGER, San Diego, CA (US); Mark ERLANDER, San Diego, CA (US)

(21) Appl. No.: 17/796,157

(22) PCT Filed: Jan. 29, 2021
(86) PCT No.: PCT/US2021/015592

§ 371 Date: Jul. 28, 2022

Related U.S. Application Data
(60) Provisional application No. 62/967,271, filed on Jan. 29, 2020.

(51) Int. Cl. A61K 31/519; A61K 31/635; A61K 45/06; A61P 35/02

(52) U.S. Cl. CPC A61K 31/519; A61K 31/635; A61K 45/06; A61P 35/02

(57) ABSTRACT

Provided include methods, compositions and kits for treating a leukemia or lymphoma in a subject. The method can comprise administrating a BCL-2 inhibitor and a PLK1 inhibitor (for example, onvansertib) to the subject in a manner sufficient to inhibit progression of the leukemia or lymphoma.

### CROSS-REFERENCE TO RELATED APPLICATIONS

This application is a U.S. national phase application under 35 U.S.C. § 371 of International Application No. PCT/US2021/015592, filed on Jan. 29, 2021 and published as WO 2021/155073 A1 on Aug. 5, 2021, which claims the benefit of priority to U.S. patent application Ser. No. 62/967,271, filed on Jan. 29, 2020; the content of each of which is incorporated herein by reference in its entirety.

### BACKGROUND

### Field

The present application generally relates to treatments for cancer. More specifically, combination therapies for treating leukemia or lymphoma that combine a BCL-2 inhibitor with a polo-like kinase 1 (PLK1) inhibitor are provided.

### Description of the Related Art

The Polo-like kinase 1 (PLK1) is the most well characterized member of the 5 members of the family of serine/threonine protein kinases and strongly promotes the progression of cells through mitosis. PLK1 performs several important functions throughout mitotic (M) phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis. PLK1 plays a key role in centrosome functions and the assembly of bipolar spindles. PLK1 also acts as a negative regulator of p53 family members leading to ubiquitination and subsequent degradation of p53/TP53, inhibition of the p73/TP73 mediated pro-apoptotic functions and phosphorylation/degradation of bora, a cofactor of Aurora kinase A. During the various stages of mitosis PLK1 localizes to the centrosomes, kinetochores and central spindle. PLK1 is a master regulator of mitosis and aberrantly overexpressed in a variety of human cancers including AML and is correlated with cellular proliferation and poor prognosis. There is a need to find effective treatment for leukemia and lymphomas in general, including for patients with relapsed or refractory leukemia and lymphoma, for example relapsed or refractory (R/R) AML.

### SUMMARY

Provided include methods, compositions and kits for treating leukemia or lymphoma. Disclosed herein include methods for treating leukemia or lymphoma. In some embodiments, a method of treating leukemia or lymphoma comprises administrating a B-cell lymphoma 2 (BCL-2) inhibitor and a Polo-like kinase 1 (PLK1) inhibitor to a subject with leukemia or lymphoma, thereby inhibiting progression of the leukemia or lymphoma. In some embodiments, the subject has leukemia. In some embodiments, the leukemia is acute myeloid leukemia (AML), myelodysplastic syndrome (MDS), or chronic myelomonocytic leukemia (CMML). In some embodiments, the leukemia is acute myeloid leukemia. In some embodiments, the leukemia is acute lymphocytic leukemia. In some embodiments, the subject has lymphoma. In some embodiments, the lymphoma is a Hodgkin lymphoma or a Non-Hodgkin lymphoma. In some embodiments, the leukemia or the lymphoma is advanced, metastatic, refractory, and/or relapsed. In some embodiments, the method the subject is human.

In some embodiments, the PLK inhibitor and the BCL-2 are co-administered simultaneously. In some embodiments, the PLK inhibitor and the BCL-2 are administered sequentially. In some embodiments, the administration of the PLK1 inhibitor is oral administration. In some embodiments, the administration of the BCL-2 inhibitor is oral administration.

In some embodiments, the inhibition of the leukemia or lymphoma progression is greater than the combined inhibition of progression caused by the BCL-2 inhibitor alone plus the PLK1 inhibitor alone. In some embodiments, the subject achieves a complete response. In some embodiments, the subject has received a prior BCL-2 inhibitor treatment. In some embodiments, the subject did not respond to treatment with the BCL-2 inhibitor alone. In some embodiments, the subject is known to be resistant to a BCL-2 inhibitor therapy.

In some embodiments, the subject has received at least one prior treatment for leukemia or lymphoma. In some embodiments, the prior treatment does not comprise the use of a BCL-2 inhibitor, a PLK inhibitor, or both. In some embodiments, the subject was in remission for leukemia or lymphoma. In some embodiments, the subject in remission for leukemia was in complete remission (CR), in CR with incomplete hematologic recovery (CRi), in morphologic leukemia-free state (MLFS), or in partial remission (PR).

In some embodiments, the BCL-2 inhibitor and the PLK1 inhibitor are each administered to the subject in a cycle of at least twice within a week. In some embodiments, the BCL-2 inhibitor and the PLK1 inhibitor are each administered to the subject in a cycle of at least five times within a week. In some embodiments, the BCL-2 inhibitor, the PLK1 inhibitor, or both are administered in a cycle of at least 7 days. In some embodiments, each cycle of treatment is at least about 21 days. In some embodiments, each cycle of treatment is from about 21 days to about 28 days. In some embodiments, the PLK1 inhibitor is administered on at least four days in the cycle. In some embodiments, the PLK1 inhibitor is not administered on at least one day in the cycle. In some embodiments, the BCL-2 inhibitor is administered daily. In some embodiments, the subject undergoes at least two cycles of the administration of the BCL-2 inhibitor and the PLK1 inhibitor.

In some embodiments, the BCL-2 inhibitor is selective and/or specific for BCL-2 inhibition. In some embodiments, the BCL-2 inhibitor is venetoclax, obatoclax, HA14-1, navitoclax, ABT-737, TW-37, AT101, sabutoclax or gambogic acid. In some embodiments, the BCL-2 inhibitor is venetoclax.

In some embodiments, the PLK1 inhibitor is selective and/or specific for PLK1. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280.

In some embodiments, the PLK1 inhibitor is onvansertib. In some embodiments, onvansertib is administered at 12 mg/m²-90 mg/m². In some embodiments, a maximum concentration (Cₘₐₓ) of onvansertib in a blood of the subject is from about 100 nmol/L to about 1500 nmol/L. In some embodiments, an area under curve (AUC) of a plot of a concentration of onvanserib in a blood of the subject over time is from about 1000 nmol/L.hour to about 400000 nmol/L.hour. In some embodiments, a time (Tₘₐₓ) to reach a maximum concentration of onvansertib in a blood of the subject is from about 1 hour to about 5 hours. In some embodiments, an elimination half-life (T_{1/2}) of onvansertib in a blood of the subject is from about 10 hours to about 60 hours. In some embodiments, the BCL-2 inhibitor is venetoclax and the PLK1 inhibitor is onvansertib.

In some embodiments, the method further comprises determining leukemia or lymphoma status of the subject. In some embodiments, the method further comprises determining responsiveness of the subject to a PLK1 inhibitor treatment. In some embodiments, the method further comprises administering one or more cancer therapeutics or therapies for leukemia or lymphoma.

Disclosed herein include kits, for example, for treating leukemia or lymphoma. In some embodiments, a kit comprises: a Polo-like kinase 1 (PLK1) inhibitor; and a manual providing instructions for co-administrating the PLK1 inhibitor with a B-cell lymphoma 2 (BCL-2) inhibitor to a subject for treating leukemia and lymphoma. In some embodiments, the kit comprises the BCL-2 inhibitor.

In some embodiments, the subject has leukemia. In some embodiments, the leukemia is acute myeloid leukemia (AML), myelodysplastic syndrome (MDS), or chronic myelomonocytic leukemia (CMML). In some embodiments, the leukemia is acute myeloid leukemia. In some embodiments, the leukemia is acute lymphocytic leukemia. In some embodiments, the subject has lymphoma. In some embodiments, the lymphoma is a Hodgkin lymphoma or a Non-Hodgkin lymphoma. In some embodiments, the leukemia or the lymphoma is advanced, metastatic, refractory, and/or relapsed.

In some embodiments, the instructions comprise instructions for co-administrating the PLK inhibitor and the BCL-2 simultaneously. In some embodiments, the instructions comprise instructions for co-administrating the PLK inhibitor and the BCL-2 sequentially. In some embodiments, the instructions comprise instructions for administering of the PLK1 inhibitor orally. In some embodiments, the instructions comprise instructions for administrating the BCL-2 inhibitor orally.

In some embodiments, the instructions comprise instructions the subject has received a prior BCL-2 inhibitor treatment. In some embodiments, the instructions comprise instructions the subject did not respond to treatment with the BCL-2 inhibitor alone. In some embodiments, the instructions comprise instructions the subject is known to be resistant to a BCL-2 inhibitor therapy.

In some embodiments, the instructions comprise instructions the subject has received at least one prior treatment for leukemia or lymphoma. In some embodiments, the prior treatment does not comprise the use of a BCL-2 inhibitor, a PLK inhibitor, or both. In some embodiments, the instructions comprise instructions the subject was in remission for leukemia or lymphoma. In some embodiments, the subject in remission for leukemia was in complete remission (CR), in CR with incomplete hematologic recovery (CRi), in morphologic leukemia-free state (MLFS), or in partial remission (PR).

In some embodiments, the instructions comprise instructions for administering each of the BCL-2 inhibitor and the PLK1 inhibitor to the subject in a cycle of at least twice within a week. In some embodiments, the instructions comprise instructions for administering each of the BCL-2 inhibitor and the PLK1 inhibitor to the subject in a cycle of at least five times within a week In some embodiments, the instructions comprise instructions for administering the BCL-2 inhibitor, the PLK1 inhibitor, or both are in a cycle of at least 7 days. In some embodiments, each cycle of treatment is at least about 21 days. In some embodiments, each cycle of treatment is from about 21 days to about 28 days. In some embodiments, the instructions comprise instructions for administering the PLK1 inhibitor on at least four days in the cycle. In some embodiments, the instructions comprise instructions for not administering the PLK1 inhibitor on at least one day in the cycle. In some embodiments, the instructions comprise instructions for administrating the BCL-2 inhibitor daily. In some embodiments, the instructions comprise instructions for administrating the BCL-2 inhibitor and the PLK1 inhibitor for at least two cycles.

In some embodiments, the BCL-2 inhibitor is selective and/or specific for BCL-2 inhibition. In some embodiments, the BCL-2 inhibitor is venetoclax, obatoclax, HA14-1, navitoclax, ABT-737, TW-37, AT101, sabutoclax or gambogic acid. In some embodiments, the BCL-2 inhibitor is venetoclax.

In some embodiments, the PLK1 inhibitor is selective and/or specific for PLK1. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280.

In some embodiments, the PLK1 inhibitor is onvansertib. In some embodiments, the instructions comprise instructions for administering onvansertib at 12 mg/m²-90 mg/m². In some embodiments, the BCL-2 inhibitor is venetoclax and the PLK1 inhibitor is onvansertib.

### BRIEF DESCRIPTION OF THE DRAWINGS

FIGS. 1A-1D are plots showing individual tumor volumes for study arms.

FIG. 2 is a survival plot for the study groups.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

The first PLK1 inhibitor, BI 2536, showed interesting clinical activity in patients with relapsed and treatment refractory AML in an early clinical study, and its successor volasertib (also known as BI 6727) demonstrated a more favorable toxicity profile, as well as potent anti-leukemic activity as monotherapy and in combination with low dose cytarabine (LDAC) in heavily pretreated AML patients. In 2013, volasertib received a Breakthrough Therapy designation from the Food and Drug Administration (FDA) for its potential as a treatment for patients with untreated AML who are ineligible for intensive remission induction therapy.

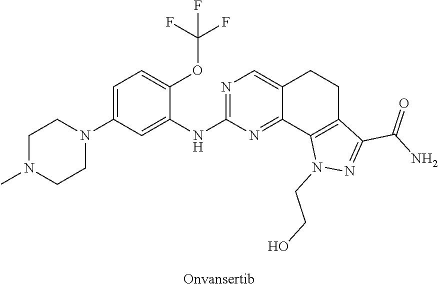 [SMILES: CN1CCN(c2ccc(OC(F)(F)F)c(Nc3ncc4c(n3)-c3c(c(C(N)=O)nn3CCO)CC4)c2)CC1]

Onvansertib (also known as PCM-075, NMS-1286937, NMS-937, “compound of formula (I)” in U.S. Patent No. 8,927,530; IUPAC name 1-(2-hydroxyethyl)-8-{[5-(4-methylpiperazin-1l-yl)-2- (trifluoromethoxy) phenyl] amino}-4,5-dihydro-1H-pyrazolo[4,3-h] quinazoline-3-carboxamide) is the first PLK1 specific ATP competitive inhibitor administered by oral route to enter clinical trials with proven antitumor activity in different preclinical models.

Onvansertib shows high potency in proliferation assays having low nanomolar activity on a large number of cell lines, both from solid as well as hematologic tumors. Onvansertib potently causes a mitotic cell-cycle arrest followed by apoptosis in cancer cell lines and inhibits xenograft tumor growth with a clear PLK1-related mechanism of action at well tolerated doses in mice after oral administration. In addition, onvansertib shows activity in combination therapy with approved cytotoxic drugs, such as irinotecan, in which there is enhanced tumor regression in HT29 human colon adenocarcinoma xenografts compared to each agent alone, and shows prolonged survival of animals in a disseminated model of AML in combination therapy with cytarabine. Onvansertib has favorable pharmacologic parameters and good oral bioavailability in rodent and nonrodent species, as well as proven antitumor activity in different nonclinical models using a variety of dosing regimens, which may potentially provide a high degree of flexibility in dosing schedules, warranting investigation in clinical settings. Onvansertib has several advantages over volasertib, including a higher degree of potency and specificity for the PLK1 isozyme, and oral bioavailability.

Onvansertib is currently in a phase 1b/2 clinical trial, having ClinicalTrials.gov Identifier NCT03303339, and entitled Onvansertib in Combination with either Low-Dose Cytarabine or Decitabine in Patients with Relapsed/Refractory Acute Myeloid Leukemia.

The BCL-2-selective inhibitor venetoclax (ABT-199) has demonstrated clinical activity in chronic lymphocytic leukemia and BCL-2 is also a target in AML and high-risk myelodysplastic syndrome where increases in anti-apoptotic BCL-2 family proteins are associated with disease progression and apoptotic resistance. Additionally, the combination of venetoclax and the PLK inhibitor volasertib has shown activity in double-hit lymphoma. However, clinical activity of venetoclax as a monotherapy is modest in AML and resistance to venetoclax treatment in AML patients has been observed. There is a need to find effective treatment for leukemia and lymphomas in general, including for patients with relapsed or refractory leukemia and lymphoma, for example relapsed or refractory (R/R) AML.

Disclosed herein include methods for treating leukemia or lymphoma. In some embodiments, a method of treating leukemia or lymphoma comprises administrating a B-cell lymphoma 2 (BCL-2) inhibitor and a Polo-like kinase 1 (PLK1) inhibitor (e.g., onvansertib) to a subject with leukemia or lymphoma, thereby inhibiting progression of the leukemia or lymphoma. Disclosed herein include kits, for example, for treating leukemia or lymphoma. In some embodiments, a kit comprises: a PLK1 inhibitor (e.g., onvansertib); and a manual providing instructions for co-administrating the PLK1 inhibitor with a BCL-2 inhibitor to a subject for treating leukemia and lymphoma.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, a “subject” refers to an animal that is the object of treatment, observation or experiment. “Animals” include cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional, such as a Medical Doctor (i.e., Doctor of Allopathic medicine or Doctor of Osteopathic medicine) or a Doctor of Veterinary Medicine, to attempt to cure, or at least ameliorate the effects of, a particular disease or disorder or to prevent the disease or disorder from occurring in the first place. In some embodiments, the patient is a human or an animal. In some embodiments, the patient is a mammal.

As used herein, “administration” or “administering” refers to a method of giving a dosage of a pharmaceutically active ingredient to a vertebrate.

As used herein, a “dosage” refers to the combined amount of the active ingredients (e.g., cyclosporine analogues, including CRV431).

As used herein, a “unit dosage” refers to an amount of therapeutic agent administered to a patient in a single dose.

As used herein, the term “daily dose” or “daily dosage” refers to a total amount of a pharmaceutical composition or a therapeutic agent that is to be taken within 24 hours.

As used herein, the term “delivery” refers to approaches, formulations, technologies, and systems for transporting a pharmaceutical composition or a therapeutic agent into the body of a patient as needed to safely achieve its desired therapeutic effect. In some embodiments, an effective amount of the composition or agent is formulated for delivery into the blood stream of a patient.

As used herein, the term “formulated” or “formulation” refers to the process in which different chemical substances, including one or more pharmaceutically active ingredients, are combined to produce a dosage form. In some embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to a diseased tissue or a tissue adjacent to the diseased tissue. Carriers or excipients can be used to produce compositions. The carriers or excipients can be chosen to facilitate administration of a drug or pro-drug. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this disclosure are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “hydrate” refers to a complex formed by combination of water molecules with molecules or ions of the solute. As used herein, the term “solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

As used herein, “therapeutically effective amount” or “pharmaceutically effective amount” refers to an amount of therapeutic agent, which has a therapeutic effect. The dosages of a pharmaceutically active ingredient which are useful in treatment when administered alone or in combination with one or more additional therapeutic agents are therapeutically effective amounts. Thus, as used herein, a therapeutically effective amount refers to an amount of therapeutic agent which produces the desired therapeutic effect as judged by clinical trial results and/or model animal studies. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treat,” “treatment,” or “treating,” refers to administering a therapeutic agent or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition. As used herein, a “therapeutic effect” relieves, to some extent, one or more of the symptoms of a disease or disorder. For example, a therapeutic effect may be observed by a reduction of the subjective discomfort that is communicated by a subject (e.g., reduced discomfort noted in self-administered patient questionnaire).

As used herein, the term “prophylaxis,” “prevent,” “preventing,” “prevention,” and grammatical variations thereof as used herein refers the preventive treatment of a subclinical disease-state in a subject, e.g., a mammal (including a human), for reducing the probability of the occurrence of a clinical disease-state. The method can partially or completely delay or preclude the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms. The subject is selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state.

As used herein, each of the terms “partial response” and “partial remission” refers to the amelioration of a cancerous state, as measured by, for example, tumor size and/or cancer marker levels, in response to a treatment. In some embodiments, a “partial response” means that a tumor or tumor-indicating blood marker has decreased in size or level by about 50% in response to a treatment. The treatment can be any treatment directed against cancer, including but not limited to, chemotherapy, radiation therapy, hormone therapy, surgery, cell or bone Marrow transplantation, and immunotherapy. The size of a tumor can be detected by clinical or by radiological means. Tumor-indicating markers can be detected by means well known to those of skill, e.g., ELISA or other antibody-based tests.

As used herein, each of the terms “complete response” or “complete remission” means that a cancerous state, as measured by, for example, tumor size and/or cancer marker levels, has disappeared following a treatment, including but are not limited to, chemotherapy, radiation therapy, hormone therapy, surgery, cell or bone marrow transplantation, and immunotherapy. The presence of a tumor can be detected by clinical or by radiological means. Tumor-indicating markers can be detected by means well known to those of skill, e.g., ELISA or other antibody-based tests. A “complete response” does not necessarily indicate that the cancer has been cured, however, as a complete response can be followed by a relapse.

### Leukemia and Lymphoma

Methods, compositions and kits disclosed herein can be used for treating leukemia or lymphoma. In some embodiments, a method for treating leukemia or lymphoma comprises administrating a B-cell lymphoma 2 (BCL-2) inhibitor, or a pharmaceutically acceptable salt, solvate, stereoisomer thereof, and a Polo-like kinase 1 (PLK1) inhibitor (e.g., onvansertib), or a pharmaceutically acceptable salt, solvate, stereoisomer thereof, to a subject (e.g., a patient) in need thereof. The method can comprise administering a pharmaceutically effective amount of the BCL-2 inhibitor and a pharmaceutically effective amount of the PLK1 inhibitor.

Leukemia is a malignant cancer of the bone marrow and blood. It is characterized by the uncontrolled growth of blood cells. The common types of leukemia are divided into four categories: acute or chronic myelogenous, involving the myeloid elements of the bone marrow (white cells, red cells, megakaryocytes) and acute or chronic lymphocytic, involving the cells of the lymphoid lineage. The most common types of leukemia in adults are acute myelogenous leukemia (AML), chronic lymphocytic leukemia (CLL), and chronic myeloid leukemia (CML). The most common type of leukemia in children is acute lymphocytic leukemia (ALL).

Acute leukemia is a rapidly progressing disease that results in the massive accumulation of immature, functionless cells (blasts) in the marrow and blood. The marrow often can no longer produce enough normal red and white blood cells and platelets. Anemia, a deficiency of red cells, develops in virtually all leukemia patients. The lack of normal white cells impairs the body's ability to fight infections. A shortage of platelets results in bruising and easy bleeding. In contrast, chronic leukemia progresses more slowly and leads to unregulated proliferation and hence marked overexpansion of a spectrum of mature (differentiated) cells. In general, acute leukemia, unlike the chronic form, is potentially curable by elimination of the neoplastic clone.

Standard treatment for leukemia usually involves chemotherapy and/or bone marrow transplantation and/or radiation therapy. The two major types of bone marrow transplants are autologus (i.e., uses the patient's own marrow) and allogeneic (i.e., uses marrow from a compatible donor). Radiation therapy, which involves the use of high-energy rays, is usually given before bone marrow transplantation to kill all leukemic cells. Chemotherapy in leukemia usually involves a combination of two or more anti-cancer drugs. Approximately 40 different drugs are now being used in the treatment of leukemia. Some common combinations include cytarabine with either doxorubicin or daunorubicin or mitoxantrone or thioguanine, mercaptopurine with methotrexate, mitroxantrone with etoposide, asparaginase with vincristine, daunorubicin and prednisone, cyclophosphamide with vincristine, cytarabine and prednisone, cyclophosphamide with vincristine and prednisone, daunorubicin with cytarabine and thioguanine and daunorubicin with vincristine and prednisone.

Acute myeloid leukemia (AML) is characterized by the clonal expansion of myeloid blasts resulting in bone marrow failure. AML is predominantly a disease of older patients with a median age at diagnosis of 68 years. For patients with AML who are believed unfit for, or do not desire, intensive treatment, hypomethylating agents (HMA; e.g., azacitidine or decitabine) or low-dose cytarabine (LDAC) have historically been treatment options. However, complete responses are uncommon and often of limited duration. In 2018, new agents (venetoclax and glasdegib) were approved in the U.S. in the first-line setting in combination with HMAs or LDAC for older and unfit patients based on phase II open-label trials. Recent updates from the ongoing randomized phase III studies showed significant increase in overall survival (OS) for venetoclax in combination with azacitidine, but not LDAC. Patients with relapsed or refractory (R/R) AML have very limited effective therapy options, particularly in the absence of targetable mutations such as FLT3 or IDH1/2, and their outcomes are dismal with median survival of less than 6 months.

Leukemia encompasses, for example, T cell leukemias or leukemias involves B cells. Examples of leukemia include, but are not limited to acute lymphoblastic leukemia (ALL, including subtypes such as precursor B acute lymphoblastic leukemia, precursor T acute lymphoblastic leukemia, Burkitt's leukemia, and acute biphenotypic leukemia), chronic lymphocytic leukemia (CLL, including for example a subtype B-cell prolymphocytic leukemia), acute myelogenous leukemia (AML, including subtypes such as acute promyelocytic leukemia, acute myeloblastic leukemia, and acute megakaryoblastic leukemia). chronic myelogenous leukemia (CML, including for example a subtype chronic myelomonocytic leukemia), hairy cell leukemia (HCL), T-cell prolymphocytic leukemia (T-PLL), large granular lymphocytic leukemia, adult T-cell leukemia, and chronic eosinophilic leukemia.

Lymphoma, also known as malignant lymphoma (ML), involves the cells of the lymphatic system. ML includes Hodgkin's lymphoma, and non-Hodgkin's lymphoma (NHL) which are a heterogeneous group of lymphoid proliferative diseases. Hodgkin's lymphoma accounts for approximately 14% of all malignant lymphomas. The non-Hodgkin's lymphomas are a diverse group of malignancies that are predominately of B-cell origin. In the Working Formulation classification scheme, these lymphomas been divided into low-, intermediate-, and high-grade categories by virtue of their natural histories. The low-grade lymphomas are indolent, with a median survival of 5 to 10 years. Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, a significant proportion of these patients will relapse and require further treatment. Examples of non-Hodgkin's lymphoma include, but are not limited to, aggressive NHL, transformed NHL, indolent NHL, relapsed NHL, refractory NHL, low grade non-Hodgkin's Lymphoma, follicular lymphoma, large cell lymphoma, B-cell lymphoma (including mature B cell lymphoma), T-cell lymphoma (including mature T cell lymphoma), Mantle cell lymphoma, Burkitt's lymphoma, NK cell lymphoma, diffuse large B-cell lymphoma, and acute lymphoblastic lymphoma.

In some embodiments, the lymphoma is a B-cell lymphoma. In some embodiments, the lymphoma is a T-cell lymphoma. In some embodiments, the lymphoma is a Hodgkins lymphoma. In some embodiments, the lymphoma is a non-Hodgkins lymphoma. In some embodiments, the lymphoma is chronic lymphocytic leukemia (CLL), small lymphocytic lymphoma (SLL), follicular lymphoma (FL), transformed follicular lymphoma (tFL), diffuse large B-cell lymphoma (DLBCL), mantle cell lymphoma (MCL), B-cell non-Hodgkin's lymphoma (NHL), peripheral T-cell lymphoma (PTCL), cutaneous T-cell lymphoma (CTCL), marginal zone lymphoma, mucosa-associated lymphoid tissue (MALT), or Waldenstrom macroglobluinemia (WM). In some embodiments, the lymphoma is a PTCL, FL, CLL or SLL.

In some embodiments, the lymphoma expresses one or more of Mcl-1, FOXP1, GAB1, SOCS1, and SOCS3 above base line. In some embodiments, base line is the expression level in a human that does not have a lymphoma. In some embodiments, the lymphoma cell genome of the patient does not comprise an activating mutation in one or more downstream genes of the JAK/STAT signaling pathway. In some embodiments, the patient has one or more mutations in FAT4, CCND3, MYOM2, ZMYM3, KMT2D, TCF3, ARID1A, and/or AXIN1. In some embodiments, the patient has one or more mutations in ZMYM3, KMT2D, and FAT4. In some embodiments, the patient further has one or more mutations in BCL2, BCL6, and/or CD79B. In some embodiments, the patient has one or more of mutations in NOTCH1, SETD2, SIGLEC10, SPEN, PCLO, TET2 (e.g., TET2^{M66L}), MK167, FAT3, KRAS, REL (e.g., REL^{I354T}), HIST1H1E (e.g., HIST1H1E^{A47V}), KMT2C, KMT2D, and/or SF3B1. In some embodiments, the patient has one or more mutations in TP53, STAT (e.g., STAT6^{S86A}), A20 (e.g., A20^{Q150R})and/or ATM. In some embodiments, the patient does not have a mutation in EP300, TP53 and/or BTK. In some embodiments, the patient does not have EP300^{S697R}, EP300^{C1247F}, TP53^{N285K}, TP53^{R273C} and/or BTK^{C481S}.

In some embodiments, the lymphoma is relapsed or refractory lymphoma. In some embodiments, the lymphoma is transformed lymphoma. In some embodiments, the lymphoma is relapsed or refractory transformed lymphoma. In some embodiments, the follicular lymphoma is relapsed or refractory follicular lymphoma. In some embodiments, the follicular lymphoma is transformed follicular lymphoma. In some embodiments, the follicular lymphoma is relapsed or refractory transformed follicular lymphoma. In some embodiments, the CLL or SLL is relapsed or refractory CLL or SLL.

### BCL-2 Inhibitors and PLK Inhibitors

Methods, compositions and kits disclosed herein can be used for treating leukemia or lymphoma. In some embodiments, a method for treating leukemia or lymphoma comprises administrating a B-cell lymphoma 2 (BCL-2) inhibitor, or a pharmaceutically acceptable salt, solvate, stereoisomer thereof, and a Polo-like kinase 1 (PLK1) inhibitor (e.g., onvansertib), or a pharmaceutically acceptable salt, solvate, stereoisomer thereof, to a subject (e.g., a patient) in need thereof. The method can comprise administering a pharmaceutically effective amount of the BCL-2 inhibitor and a pharmaceutically effective amount of the PLK1 inhibitor.

BCL-2 proteins regulate programmed cell death triggered by developmental cues and in response to multiple Stress signals. BCL-2 proteins play a role in many diseases, particularly in cancer, leukemia, immune and autoimmune diseases. BCL-2 proteins are said to be involved in many types of cancer, including bone marrow cancer, chronic lymphocytic leukemia, lymphoblastic leukemia, follicular lymphoma, lymphoid malignancies of T-cell or B-cell origin, and myelogenous leukemia. Overexpression of BCL-2 proteins correlate with resistance to chemotherapy, clinical outcome, disease progression, overall prognosis or a combination thereof in various cancers and disorders of the immune system. BCL-2 inhibitors are agents (including small molecule compounds, nucleotides (e.g., antisense oligonucleotides), and proteins (e.g., antibodies)) that are capable of inhibiting (partially or completely) activities of the BCL-2 proteins. In some embodiments, the BCL-2 inhibitor prevents activity of BCL-2 proteins with an IC₅₀ of about 0.001 μM to about 2 μM. In some embodiments, the BCL-2 inhibitor is a BCL-2 selective inhibitor.

A number of BCL-2 inhibitors have been identified, including but are not limited to: oblimersen (also known as G3139 and Genasense), SPC-2996, RTA-402, gossypol (also known as AT-101), apogossypol, obatoclax mesylate, A-371191, A-385358, A-438744, ABT-737, ABT-263, AT-101, BL-11, BL-193, GX-15-003, 2-Methoxyantimycin A3, HA-14-1. KF-67544, Purpurogallin, TP-TW-37, YC-137, Z-24, venetoclax (also known as ABT-199 and GDC-0199), navitoclax (also known as ABT-263), obatoclax, sabutoclax, S-055746, antimycin A, S44563, and PNT-2258. In some embodiments, the BCL-2 inhibitor is venetoclax.

Polo-like kinases (PLK) are a family of five highly conserved serine/threonine protein kinases. PLK1 is a master regulator of mitosis and is involved in several steps of the cell cycle, including mitosis entry, centrosome maturation, bipolar spindle formation, chromosome separation, and cytokinesis. PLK1 has been shown to be overexpressed in solid tumors and hematologic malignancies, including AML. PLK1 inhibition induces G2—M-phase arrest with subsequent apoptosis in cancer cells, and has emerged as a promising targeted therapy. Several PLK inhibitors have been studied in clinical trials. In a randomized phase II study of patients with AML who were treatment naïve yet unsuitable for induction therapy, the pan-PLK inhibitor, volasertib (BI6727), administered intravenously in combination with LDAC showed a significant increase in OS when compared with LDAC alone. A subsequent randomized phase III study identified no benefit of the combination and described an increased risk of severe infections.

Onvansertib (also known as PCM-075 or NMS-1286937) is a selective ATP-competitive PLK1 inhibitor. Biochemical assays demonstrated high specificity of onvansertib for PLK1 among a panel of 296 kinases, including other PLK members. Onvansertib has potent in vitro and in vivo antitumor activity in models of both solid and hematologic malignancies. Onvansertib inhibited cell proliferation at nanomolar concentrations in AML cell lines and tumor growth in xenograft models of AML. In addition, onvansertib significantly increased cytarabine antitumor activity in disseminated models of AML.

A phase I, first-in-human, dose-escalation study of onvansertib in patients with advanced/metastatic solid tumors identified neutropenia and thrombocytopenia as the primary dose-limiting toxicities. These hematologic toxicities were anticipated on the basis of the mechanism of action of the drug and were reversible, with recovery occurring within 3 weeks. The half-life of onvansertib was established between 20 and 30 hours. The oral bioavailability of onvansertib plus its short half-life provide the opportunity for convenient, controlled, and flexible dosing schedules with the potential to minimize toxicities and improve the therapeutic window. Pharmacodynamics and biomarker studies, including baseline genomic profiling, serial monitoring of mutant allele fractions in plasma, and the extent of PLK1 inhibition in circulating blasts, have been performed to identify biomarkers associated with clinical response and are described in PCT Application No. PCT/US2021/013287, the content of which is incorporated herein by reference in its entirety.

As disclosed herein, combinations of a BCL-2 inhibitor and a PLK1 inhibitor can result in significantly enhanced efficacy against leukemias and lymphomas, causing tumor regression and cancer survival. Surprisingly, the resulted tumor regression and cancer survival rate/duration by the combination is more than additive, i.e., superior to the cumulated anti-tumor efficacy caused by the BCL-2 inhibitor and the PLK1 inhibitor separately. Provided herein include methods, compositions and kits for treating a leukemia or lymphoma in a subject (for example, a human patient suffering from leukemia or lymphoma). The method comprises administrating a BCL-2 inhibitor and a PLK1 inhibitor to the patient in a manner sufficient to inhibit progression of the leukemia or lymphoma. For example, the BCL-2 inhibitor and the PLK1 inhibitor can be administrated to a subject with leukemia or lymphoma simultaneously, separately, or sequentially.

In some embodiments, the inhibition of the leukemia or lymphoma progression is enhanced or synergistic, i.e., is greater than the combined inhibition of progression caused by the BCL-2 inhibitor alone plus the PLK1 inhibitor alone. The enhanced or synergistic efficacy or inhibition of any combination of a BCL-2 inhibitor and a PLK1 inhibitor of the present disclosure can be different in different embodiments. In some embodiments, the enhanced or synergistic efficacy or inhibition of any combination of a BCL-2 inhibitor and a PLK1 inhibitor of the present disclosure is, is about, is at least, is at least about, is at most, or is at most about, 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or a combination thereof, higher than the combined inhibition of progression caused by the BCL-2 inhibitor alone plus the PLK1 inhibitor alone.

This method is expected to be effective with any leukemia or lymphoma. Nonlimiting examples include acute myeloid leukemia (AML), acute lymphoblastic leukemia (ALL), adult T cell leukemia/lymphoma, chronic myelogenous leukemia, chronic lymphocytic leukemia, hairy cell leukemia, Hodgkin lymphoma, non-Hodgkin lymphoma, and B cell lymphoma. In some embodiments, the leukemia or lymphoma is AML.

As used herein, a patient is treated for a leukemia or lymphoma sufficient to inhibit progression of the leukemia or lymphoma if the patient achieves complete response or partial response after treatment with the BCL-2 inhibitor and the PLK1 inhibitor. Complete response is defined as a morphologic leukemia- or lymphoma-free state. In AML, complete response means bone marrow (BM) having less than 5% blasts in an aspirate with spicules and no blasts with Auer rods or persistence of extramedullary disease. Partial response is defined as all the hematologic values for a complete response but with a decrease in presence of disease. In AML, at least 50% in the percentage of blasts to 5% to 25% in the bone marrow aspirate and a normalization of blood counts. In various embodiments, the patient achieves a complete response. In other embodiments, the patient achieves a partial response. In further embodiments, the patient did not respond to treatment with the BCL-2 inhibitor alone.

The BCL-2 inhibitor and the PLK1 inhibitor can be administered to the patient in any manner deemed effective to treat the leukemia or lymphoma. The BCL-2 inhibitor can be administered together with, or separately from, the PLK1 inhibitor. When administered separately, the BCL-2 inhibitor can be administered before or after the PLK1 inhibitor, or in different administration cycles, e.g., as in Example 1 below, where the BCL-2 inhibitor (venetoclax) was given daily for two weeks and the PLK1 inhibitor (onvansertib) was given 5 days on, 2 off for two weeks.

The BCL-2 inhibitor and the PLK1 inhibitor can each be administered in any schedule, e.g., once or multiple times per day or week; once, twice, three times, four times, five times, six times or seven times (daily) per week; for one or multiple weeks; etc. In some embodiments, the BCL-2 inhibitor and the PLK1 inhibitor are each administered to the patient in a cycle of at least twice within a week. In other embodiments, the BCL-2 inhibitor and the PLK1 inhibitor are each administered to the patient in a cycle of at least five times within a week. In further embodiments, the patient undergoes at least two cycles of administration.

Any BCL-2 inhibitor, now known or later discovered, can be used in these methods, including BCL-2 inhibitors that are selective for BCL-2, and BCL-2 inhibitors that also inhibit the activity of other proteins. Nonlimiting examples of BCL-2 inhibitors are venetoclax, obatoclax, HA14-1, navitoclax, ABT-737, TW-37, AT101, sabutoclax, gambogic acid, ABT737, ABT263, gossypol, epigallocatechin gallate, licochalcone A, EM20-25, YC137, ABT263, 2,3-DCPE, nilotinib, 2-methoxy-antimycin A3, AG1024, piperlongumine, and combinations thereof. In some embodiments, the BCL-2 inhibitor is venetoclax.

Similarly, any PLK1 inhibitor, now known or later discovered, can be used in these methods, including PLK1 inhibitors that are selective for PLK1, and PLK1 inhibitors that also inhibit the activity of other proteins. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or a combination thereof. In some of these embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280.

In some embodiments, the PLK1 inhibitor is onvansertib. In these embodiments, the onvansertib is administered to the patient at any appropriate dosage, e.g., a dosage of less than 12 mg/m², less than or equal to 24 mg/m², or greater than 24 mg/m². In particular embodiments, the onvansertib is administered to the patient daily. In additional embodiments, the onvansertib is administered in a cycle of 3-10 days of daily onvansertib administration with 2-16 days with no onvansertib administration.

As can be appreciated by one of skill in the art, the amount of co-administration of the BCL-2 inhibitor and the PLK1 inhibitor, and the timing of co-administration, can depend on the type (species, gender, age, weight, etc.) and condition of the subject being treated and the severity of the disease or condition being treated. The BCL-2 inhibitor and the PLK1 inhibitor can formulated into a single pharmaceutical composition, or two separate pharmaceutical compositions. The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Methods, compositions, kits and systems disclosed herein can be applied to different types of subjects. For example, the subject can be a subject receiving a cancer treatment (e.g., for leukemia or lymphoma), a subject at cancer remission (e.g., remission for leukemia or lymphoma), a subject has received one or more cancer treatment, or a subject suspected of having cancer (e.g., leukemia or lymphoma). The subject can have a stage I cancer, a stage II cancer, a stage III cancer, and/or a stage IV cancer. The cancer can comprise a hematological cancer, for example leukemia. Non-limiting examples of leukemia include acute lymphocytic leukemia (ALL), acute myeloid leukemia (AML), chronic lymphocytic (CLL), chronic myeloid (CML), chronic myelomonocytic (CMML), and a combination thereof. The methods can comprise: administering a therapeutic intervention to the subject. The therapeutic intervention can comprise a different therapeutic intervention, an antibody, an adoptive T cell therapy, a chimeric antigen receptor (CAR) T cell therapy, an antibody-drug conjugate, a cytokine therapy, a cancer vaccine, a checkpoint inhibitor, radiation therapy, surgery, a chemotherapeutic agent, or any combination thereof. The therapeutic intervention can be administered at a time when the subject has an early-stage cancer, and wherein the therapeutic intervention is more effective that if the therapeutic intervention were to be administered to the subject at a later time.

### BCL-2 Inhibitors and PLK1 Inhibitors Dosing and Pharmacokinetics

The treatment of the present disclosure can comprise daily administration of a BCL-2 inhibitor (e.g., venetoclax) at or at about 20 mg, 50 mg, 100 mg, 200 mg, 300 mg, 400 mg, 500 mg, 600 mg, 700 mg, 800 mg, 900 mg, 1000 mg, 1100 mg, 1200 mg, or a number or a range between any two of these values. In some embodiments, the daily dose of the BCL-2 inhibitor (e.g., venetoclax) can be adjusted (e.g., increased or decreased with the range) during the treatment of the subject. The daily administration of the BCL-2 inhibitor can be at different amounts on different days or during different weeks. For example, the treatment can comprise daily administration of the BCL-2 inhibitor (e.g., venetoclax) at 20 mg during week 1, 50 mg during week 2, 100 mg during week 3, 200 mg during week 4, and 400 mg during week 5 and beyond. For example, the treatment can comprise daily administration of the BCL-2 inhibitor (e.g., venetoclax) at 100 mg on day 1, 200 mg on day 2, 400 mg on day 3, and 400 mg or 600 mg on day 4 and beyond.

A maximum concentration (Cₘₐₓ) of the BCL-2 inhibitor (e.g., venetoclax) in a blood of the subject (during the treatment or after the treatment) when the BCL-2 inhibitor is administered alone or in combination with the PLK1 inhibitor can be from about 0.1 mcg/mL to about 10 mcg/mL. For example, the Cₘₐₓ of the BCL-2 inhibitor (e.g., venetoclax) in a blood of the subject when the BCL-2 inhibitor is administered alone or in combination with the PLK1 inhibitor can be, or be about, 0.1 μg/mL, 0.2 μg/mL, 0.3 μg/mL, 0.4 μg/mL, 0.5 μg/mL, 0.6 μg/mL, 0.7 μg/mL, 0.8 μg/mL, 0.9 μg/mL, 1 μg/mL, 1.1 μg/mL, 1.2 μg/mL, 1.3 μg/mL, 1.4 μg/mL, 1.5 μg/mL, 1.6 μg/mL, 1.7 μg/mL, 1.8 μg/mL, 1.9 μg/mL, 2 μg/mL, 2.1 μg/mL, 2.2 μg/mL, 2.3 μg/mL, 2.4 μg/mL, 2.5 μg/mL, 2.6 μg/mL, 2.7 μg/mL, 2.8 μg/mL, 2.9 μg/mL, 3 μg/mL, 3.1 μg/mL, 3.2 μg/mL, 3.3 μg/mL, 3.4 μg/mL, 3.5 μg/mL, 3.6 μg/mL, 3.7 μg/mL, 3.8 μg/mL, 3.9 μg/mL, 4 μg/mL, 4.1 μg/mL, 4.2 μg/mL, 4.3 μg/mL, 4.4 μg/mL, 4.5 μg/mL, 4.6 μg/mL, 4.7 μg/mL, 4.8 μg/mL, 4.9 μg/mL, 5 μg/mL, 5.1 μg/mL, 5.2 μg/mL, 5.3 μg/mL, 5.4 μg/mL, 5.5 μg/mL, 5.6 μg/mL, 5.7 μg/mL, 5.8 μg/mL, 5.9 μg/mL, 6 μg/mL, 6.1 μg/mL, 6.2 μg/mL, 6.3 μg/mL, 6.4 μg/mL, 6.5 μg/mL, 6.6 μg/mL, 6.7 μg/mL, 6.8 μg/mL, 6.9 μg/mL, 7 μg/mL, 7.1 μg/mL, 7.2 μg/mL, 7.3 μg/mL, 7.4 μg/mL, 7.5 μg/mL, 7.6 μg/mL, 7.7 μg/mL, 7.8 μg/mL, 7.9 μg/mL, 8 μg/mL, 8.1 μg/mL, 8.2 μg/mL, 8.3 μg/mL, 8.4 μg/mL, 8.5 μg/mL, 8.6 μg/mL, 8.7 μg/mL, 8.8 μg/mL, 8.9 μg/mL, 9 μg/mL, 9.1 μg/mL, 9.2 μg/mL, 9.3 μg/mL, 9.4 μg/mL, 9.5 μg/mL, 9.6 μg/mL, 9.7 μg/mL, 9.8 μg/mL, 9.9 μg/mL, 10 μg/mL, a range between any two of these values, or any value between 0.1 mcg/mL to 10 mcg/mL.

An area under curve (AUC) of a plot of a concentration of the BCL-2 inhibitor (e.g., venetoclax) in a blood of the subject over time (e.g., AUC₀₋₂₄ for the first 24 hours after administration) when the BCL-2 inhibitor is administered alone or in combination with the PLK1 inhibitor can be from about 10 μg.h/mL to about 100 μg.h/mL. For example, the AUC of a plot of a concentration of the BCL-2 inhibitor (e.g., venetoclax) in a blood of the subject over time (e.g., AUC₀₋₂₄ for the first 24 hours after administration) when the BCL-2 inhibitor is administered alone or in combination with the PLK1 inhibitor can be, or be about, 1 μg.h/mL, 2 μg.h/mL, 3 μg.h/mL, 4 μg.h/mL, 5 μg.h/mL, 6 μg.h/mL, 7 μg.h/mL, 8 μg.h/mL, 9 μg.h/mL, 10 μg.h/mL, 11 μg.h/mL, 12 μg.h/mL, 13 μg.h/mL, 14 μg.h/mL, 15 μg.h/mL, 16 μg.h/mL, 17 μg.h/mL, 18 μg.h/mL, 19 μg.h/mL, 20 μg.h/mL, 21 μg.h/mL, 22 μg.h/mL, 23 μg.h/mL, 24 μg.h/mL, 25 μg.h/mL, 26 μg.h/mL, 27 μg.h/mL, 28 μg.h/mL, 29 μg.h/mL, 30 μg.h/mL, 31 μg.h/mL, 32 μg.h/mL, 33 μg.h/mL, 34 μg.h/mL, 35 μg.h/mL, 36 μg.h/mL, 37 μg.h/mL, 38 μg.h/mL, 39 μg.h/mL, 40 μg.h/mL, 41 μg.h/mL, 42 μg.h/mL, 43 μg.h/mL, 44 μg.h/mL, 45 μg.h/mL, 46 μg.h/mL, 47 μg.h/mL, 48 μg.h/mL, 49 μg.h/mL, 50 μg.h/mL, 51 μg.h/mL, 52 μg.h/mL, 53 μg.h/mL, 54 μg.h/mL, 55 μg.h/mL, 56 μg.h/mL, 57 μg.h/mL, 58 μg.h/mL, 59 μg.h/mL, 60 μg.h/mL, 61 μg.h/mL, 62 μg.h/mL, 63 μg.h/mL, 64 μg.h/mL, 65 μg.h/mL, 66 μg.h/mL, 67 μg.h/mL, 68 μg.h/mL, 69 μg.h/mL, 70 μg.h/mL, 71 μg.h/mL, 72 μg.h/mL, 73 μg.h/mL, 74 μg.h/mL, 75 μg.h/mL, 76 μg.h/mL, 77 μg.h/mL, 78 μg.h/mL, 79 μg.h/mL, 80 μg.h/mL, 81 μg.h/mL, 82 μg.h/mL, 83 μg.h/mL, 84 μg.h/mL, 85 μg.h/mL, 86 μg.h/mL, 87 μg.h/mL, 88 μg.h/mL, 89 μg.h/mL, 90 μg.h/mL, 91 μg.h/mL, 92 μg.h/mL, 93 μg.h/mL, 94 μg.h/mL, 95 μg.h/mL, 96 μg.h/mL, 97 μg.h/mL, 98 μg.h/mL, 99 μg.h/mL, 100 μg.h/mL, a range between any two of these values, or any value between 10 μg.h/mL and 100 μg.h/mL.

A time (Tₘₐₓ) to reach a maximum concentration of the BCL-2 inhibitor (e.g., venetoclax) in a blood of the subject when the BCL-2 inhibitor is administered alone or in combination with the PLK1 inhibitor can be from about 3 hours to 10 hours. For example, the time (Tₘₐₓ) to reach a maximum concentration of the BCL-2 inhibitor (e.g., venetoclax) in a blood of the subject when the BCL-2 inhibitor is administered alone or in combination with the PLK1 inhibitor can be, or be about, 3 hours, 4 hours, 5 hours, 6 hours, 7 hours, 8 hours, 9 hours, 10 hours, a range between any two of these values, or any value between 3 hours and 10 hours.

An elimination half-life (T_{1/2}) of the BCL-2 inhibitor (e.g., venetoclax) in a blood of the subject when the BCL-2 inhibitor is administered alone or in combination with the PLK1 inhibitor can be from about 15 hours to about 60 hours. For example, the elimination half-life (T_{1/2}) of the BCL-2 inhibitor (e.g., venetoclax) in a blood of the subject when the BCL-2 inhibitor is administered alone or in combination with the PLK1 inhibitor can be, or be about, 15 hours, 20 hours, 25 hours, 30 hours, 35 hours, 40 hours, 45 hours, 50 hours, 55 hours, 60 hours, a range between any two of these values, or any value between 15 hours and 60 hours.

The treatment of the present disclosure can comprise administration of a PLK1 inhibitor (onvansertib) for a desired duration in a cycle. The desired duration can be one, two, three, four, five, six, seven, eight, nine, ten, or more days. The cycle can be, for example, at least 20 days, 21 days, 22 days, 23 days, 24 days, 25 days, or more. For example, a single cycle of the treatment can comprise administration of the PLK1 inhibitor (e.g., onvansertib) for four days, five days, six days, seven days, eight days, nine days, ten days, eleven days, twelve days, thirteen days, fourteen days, fifteen days, sixteen days, seventeen days, eighteen days, nineteen days, twenty days, or more in a cycle (e.g., a cycle of at least 21 days (e.g., 21 to 28 days)). In some embodiments, the treatment can comprise administration of the PLK1 inhibitor (e.g., onvansertib) for, or for at least, four days, five days, six days, seven days, eight days, nine days, ten days, eleven days, twelve days, thirteen days, fourteen days, fifteen days, sixteen days, seventeen days, eighteen days, nineteen days, twenty days, or a range between any two of these values, in a cycle (e.g., a cycle of at least 21 days (e.g., 21 to 28 days)). The administration of the PLK1 inhibitor (e.g., onvansertib) in a single cycle of the treatment can be continuous or with one or more intervals (e.g., one day or two days of break). In some embodiments, the treatment comprises administration of the PLK1 inhibitor (e.g., onvansertib) for five days in a cycle of 21 to 28 days.

The treatment can comprise administration of the PLK1 inhibitor (e.g., onvansertib) at, or at about, 12 mg/m²-90 mg/m², for example, as a daily dose. For example, the treatment can comprise daily administration of the PLK1 inhibitor (e.g., onvansertib) at, or at about, 8 mg/m²,10 mg/m²,12 mg/m²,14 mg/m²,16 mg/m²,18 mg/m²,20 mg/m²,23 mg/m²,27 mg/m²,30 mg/m²,35 mg/m²,40 mg/m²,45 mg/m²,50 mg/m²,55 mg/m²,60 mg/m²,65 mg/m², 70 mg/m²,80 mg/m²,85 mg/m²,90 mg/m², a range between any two of these values, or any value between 8 mg/m²-90 mg/m². In some embodiments, the daily dose of the PLK1 inhibitor (e.g., onvansertib) can be adjusted (e.g., increased or decreased with the range) during the treatment, or during a single cycle (e.g., the first cycle, the second cycle, the third cycle, and a subsequent cycle) of the treatment, for the subject.

A maximum concentration (Cₘₐₓ) of the PLK1 inhibitor (e.g., onvansertib) in a blood of the subject (during the treatment or after the treatment) when the PLK1 inhibitor is administered alone or in combination with the BCL-2 inhibitor can be from about 100 nmol/L to about 1500 nmol/L. For example, the Cₘₐₓ of the PLK1 inhibitor (e.g., onvansertib) in a blood of the subject when the PLK1 inhibitor is administered alone or in combination with the BCL-2 inhibitor can be, or be about, 100 nmol/L, 200 nmol/L, 300 nmol/L, 400 nmol/L, 500 nmol/L, 600 nmol/L, 700 nmol/L, 800 nmol/L, 900 nmol/L, 1000 nmol/L, 1100 nmol/L, 1200 nmol/L, 1300 nmol/L, 1400 nmol/L, 1500 nmol/L, a range between any two of these values, or any value between 200 nmol/L to 1500 nmol/L.

An area under curve (AUC) of a plot of a concentration of the PLK1 inhibitor (e.g., onvanserib) in a blood of the subject over time (e.g., AUC₀₋₂₄ for the first 24 hours after administration) when the PLK1 inhibitor is administered alone or in combination with the BCL-2 inhibitor can be from about 1000 nmol/L.hour to about 400000 nmol/L.hour. For example, the AUC of a plot of a concentration of the PLK1 inhibitor (e.g., onvansertib) in a blood of the subject over time (e.g., AUC₀₋₂₄ for the first 24 hours after administration) when the PLK1 inhibitor is administered alone or in combination with the BCL-2 inhibitor can be, or be about, 1000 nmol/L.hour, 5000 nmol/L.hour, 10000 nmol/L.hour, 15000 nmol/L.hour, 20000 nmol/L.hour, 25000 nmol/L.hour, 30000 nmol/L.hour, 35000 nmol/L.hour, 40000 nmol/L.hour, a range between any two of these values, or any value between 1000 nmol/L.hour and 400000 nmol/L.hour.

A time (Tₘₐₓ) to reach a maximum concentration of the PLK1 inhibitor (e.g., onvansertib) in a blood of the subject when the PLK1 inhibitor is administered alone or in combination with the BCL-2 inhibitor can be from about 1 hour to about 5 hours. For example, the time (Tₘₐₓ) to reach a maximum concentration of the PLK1 inhibitor (e.g., onvansertib) in a blood of the subject when the PLK1 inhibitor is administered alone or in combination with the BCL-2 inhibitor can be, or be about, 1 hour, 1.5 hours, 2 hours, 2.5 hours, 3 hours, 3.5 hours, 4 hours, 4.5 hours, 5 hours, a range between any two of these values, or any value between 1 hour and 5 hours.

An elimination half-life (T_{1/2}) of the PLK1 inhibitor (e.g., onvansertib) in a blood of the subject when the PLK1 inhibitor is administered alone or in combination with the BCL-2 inhibitor can be from about 10 hours to about 60 hours. For example, the elimination half-life (T_{1/2}) of the PLK1 inhibitor (e.g., onvansertib) in a blood of the subject when the PLK1 inhibitor is administered alone or in combination with the BCL-2 inhibitor can be, or be about, 10 hours, 15 hours, 20 hours, 25 hours, 30 hours, 35 hours, 40 hours, 45 hours, 50 hours, 55 hours, 60 hours, a range between any two of these values, or any value between 10 hours and 60 hours.

### Additional Cancer Therapeutics or Therapy

Methods, compositions and kits disclosed herein can be used for treating leukemia or lymphoma. In some embodiments, a method for treating leukemia or lymphoma comprises administrating a BCL-2 inhibitor and a PLK1 inhibitor (e.g., onvansertib) to a subject (e.g., a patient) in need thereof. The method can comprise administering a therapeutically effective amount of the BCL-2 inhibitor and a therapeutically effective amount of the PLK1 inhibitor. The treatment can comprise administration of at least one additional cancer therapeutics or cancer therapy. The treatment can comprise administration a therapeutically effective amount of at least one additional cancer therapeutics or cancer therapy. The BCL-2 inhibitor and the cancer therapeutics or cancer therapy can, for example, co-administered simultaneously or sequentially. The PLK1 inhibitor (e.g., onvansertib) and the cancer therapeutics or cancer therapy can, for example, co-administered simultaneously or sequentially. In some embodiments, the additional cancer therapeutics is cytarabine, low-dose cytarabine (LDAC) and/or decitabine. The safety, pharmacokinetics, and preliminary clinical activity of onvansertib in combination with either LDAC or decitabine have been determined in patients with R/R AML and are described in PCT Application No. PCT/US2021/013287, the content of which is incorporated herein by reference in its entirety. In some embodiments, the treatment comprises administration of LDAC at, or at about, 20 mg/m² subcutaneous (SC) once a day (qd) for seven, eight, night, ten, eleven, twelve, or thirteen days in a cycle. In some embodiments, the treatment comprises administration of decitabine at, or at about, 20 mg/m² intravenous (IV) qd for three, four, five, six, or seven days in a cycle. In some embodiments, the treatment comprises administration of LDAC at, or at about, 20 mg/m² subcutaneous (SC) once a day (qd) for ten days in a cycle, and administration of decitabine at 20 mg/m² intravenous (IV) qd for five days in a cycle.

### Methods for Predicting/Determining Treatment Efficacy and Status for Cancer

Also disclosed herein include methods, compositions, kits, and systems for predicting/determining clinical outcome for a combination treatment of leukemia or lymphoma of the present disclosure, monitoring of the combination treatment, predicting/determining responsiveness of a subject to the combination treatment, determining the status of the leukemia or lymphoma in a subject, and improving combination treatment outcome. The methods, compositions, kits and systems can be used to guide the combination treatment, provide combination treatment recommendations, reduce or avoid unnecessary ineffective combination treatment for patients. ctDNA can be analyzed to predict/determine clinical outcome for leukemia or lymphoma treatment using a combination of a BCL-2 inhibitor and a PLK1 inhibitor of the present disclosure, monitor the combination treatment, predict/determine responsiveness of a subject to the combination treatment, determine leukemia or lymphoma status in a subject, improve combination treatment outcome, guide combination treatment, provide combination treatment recommendations, and/or to reduce or avoid ineffective combination treatment. ctDNA can be analyzed to predict/determine clinical outcome for cancer treatment, monitor cancer treatment, predict/determine responsiveness of a subject to a cancer treatment, determine cancer status in a subject, improve cancer treatment outcome, guide cancer treatment, provide treatment recommendations, and/or to reduce or avoid ineffective cancer treatment. Such analysis of ctDNA has been described in PCT Application No. PCT/US2021/013287, the content of which is incorporated herein by reference in its entirety.

A method of determining responsiveness of a subject to a combination treatment comprising a BCL-2 inhibitor and a PLK1 inhibitor of the disclosure can comprise, for example, analyzing circulating tumor DNA (ctDNA) of a subject with leukemia or lymphoma, the subject is undergoing a treatment and/or has received the combination treatment, thereby determining the responsiveness of the subject to the combination treatment. In some embodiments, determining the responsiveness of the subject comprises determining if the subject is a responder of the treatment, if the subject is or is going to be in CR, if the subject is or is going to be in incomplete hematologic recovery (CRi), if the subject is or is going to be in morphologic leukemia-free state (MLFS), or if the subject is or is going to be in partial remission (PR). For example, analyzing ctDNA can comprise detecting variant allele frequency in the ctDNA in a first sample obtained from the subject at a first time point, detecting variant allele frequency in the ctDNA obtained from the subject at one or more additional time points in one or more additional samples, and determining the difference of the variant allele frequency in ctDNA between the first and at least one of the one or more additional samples, a decrease in the variant allele frequency in at least one of the additional samples relative to the first sample indicates the subject as responsive to the leukemia treatment.

In some embodiments, the first time point is prior or immediately prior to the combination treatment, and at least one of the one or more additional time points are at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment.

In some embodiments, the first cycle of the combination treatment is immediately prior to the second cycle of the combination treatment. In some embodiments, the method comprises continuing the combination treatment to the subject if the subject is indicated as responsive to the combination treatment. In some embodiments, the method comprises discontinuing the combination treatment to the subject and/or starting a different combination treatment to the subject if the subject is not indicated as responsive to the combination treatment.

Disclosed herein include methods of determining leukemia or lymphoma status of a subject, comprising analyzing circulating tumor DNA (ctDNA) of a subject, thereby determining leukemia or lymphoma status of the subject. The subject can be a subject undergoing a current combination treatment comprising a BCL-2 inhibitor and a PLK1 inhibitor of the present disclosure, a subject that has received a prior combination treatment of the present disclosure, and/or a subject that is in remission for leukemia or lymphoma. The subject in remission for leukemia or lymphoma can be in complete remission (CR), in CR with incomplete hematologic recovery (CRi), in morphologic leukemia-free state (MLFS), or in partial remission (PR).

In some embodiments, analyzing the ctDNA comprises detecting variant allele frequency in the ctDNA. In some embodiments, analyzing the ctDNA comprises detecting variant allele frequency in the ctDNA obtained from the subject at a first time point in a first sample, detecting variant allele frequency in the ctDNA obtained from the subject at one or more additional time points in one or more additional samples, and determining the difference of the variant allele frequency in ctDNA between the first and at least one of the one or more additional samples, an increase in the variant allele frequency at the additional sample(s) relative to the first sample indicates that the subject is at risk of leukemia or lymphoma relapse or is in leukemia or lymphoma relapse.

In some embodiments, the first time point is prior or immediately prior to the combination treatment, and the one or more additional time points are at the end of or after at least a cycle of the combination treatment, optionally the cycle of the combination treatment is the first cycle of the combination treatment. In some embodiments, the first time point is prior or immediately prior to a first cycle of the combination treatment, and the one or more additional time points are at the end of or after a second cycle of the combination treatment, optionally the first cycle of the combination treatment is immediately prior to the second cycle of the combination treatment.

In some embodiments, the method comprises starting an additional treatment to the subject if the subject is indicated as in leukemia or lymphoma relapse. The additional treatment can be the same or different from the current or prior combination treatment.

The variant allele frequency in ctDNA can be determined, for example, by total mutation count in the ctDNA in each of the first sample and one or more additional samples, or by the mean variant allele frequency in each of the first sample and one or more additional samples. In some embodiments, the variant allele frequency is mutant allelic frequency (MAF) for a driver mutation of leukemia. In some embodiments, the variant allele frequency is MAF for one or more driver mutations of leukemia. In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is, or is about, 0.01 to −0.10. In some embodiments, the MAF threshold is, or is about, 0.06. In some embodiments, the MAF threshold is, or is about, 0.05.

In some embodiments, the first sample comprises ctDNA from the subject before treatment, and the one of additional samples comprises ctDNA from the subject after treatment. In some embodiments, the driver mutation is a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PHF6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2. In some embodiments, at least one of the one or more the driver mutations is a mutation in in the 75 genes. In some embodiments, one or more the driver mutations are mutations in the 75 genes.

The driver mutation or at least one of the one or more driver mutations can be in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1. In some embodiments, the method further comprises determining variant allele frequency in one or more of the ctDNA, PBMCs and BMMCs of the subject.

The ctDNA can be analyzed using, for example, polymerase chain reaction (PCR), next generation sequencing (NGS), and/or droplet digital PCR (ddPCR). The sample disclosed herein can be derived from, for example, whole blood of the subject, plasma of the subject, serum of the subject, or a combination thereof. In some embodiments, the ctDNA is from whole blood of the subject, plasma of the subject, serum of the subject, or a combination thereof.

In some embodiments, the method comprises analyzing ctDNA of the subject before the treatment. In some embodiments, the treatment comprises one or more cycles, and the ctDNA is analyzed before, during and after each cycle of the treatment. Each cycle of treatment can be at least 21 days. In some embodiments, each cycle of treatment is from about 21 days to about 28 days. In some embodiments, the subject is human.

Disclosed herein include methods of improving treatment outcome for leukemia or lymphoma. The method can comprise: detecting variant allele frequency in circulating tumor DNA (ctDNA) obtained from a subject at a first time point in a first sample before the subject undergoes a combination treatment comprising a BCL-2 inhibitor and a PLK1 inhibitor of the present disclosure; detecting variant allele frequency in ctDNA obtained from the subject at one or more additional time points in one or more additional samples after the subject undergoes the combination treatment; determining the difference of the variant allele frequency in ctDNA between the first and at least one of the one or more additional samples, a decrease in the variant allele frequency in at least one of the additional samples relative to the first sample indicates the subject as responsive to the combination treatment; and continuing the combination treatment to the subject if the subject is indicated as responsive to the combination treatment, or discontinuing the combination treatment to the subject and/or starting a different leukemia or lymphoma treatment to the subject if the subject is not indicated as responsive to the combination treatment.

Also disclosed herein include methods of treating leukemia or lymphoma. The method can comprise: administering a combination treatment comprising a BCL-2 inhibitor and a PLK1 inhibitor of the present disclosure to a subject in need thereof; determining a decrease, relative to a variant allele frequency in a first sample of the subject obtained at a first time point before the subject receives the combination treatment, in a variant allele frequency in a second sample of the subject obtained at a second time point after the subject receives the combination treatment; and continuing with the combination treatment. In some embodiments, the subject is a subject newly diagnosed with leukemia or lymphoma, for example a subject that has not received any prior leukemia or lymphoma treatment before the combination treatment. In some embodiments, the subject has received prior leukemia or lymphoma treatment and was in remission for leukemia or lymphoma, for example a subject in complete remission (CR), in CR with incomplete hematologic recovery (CRi), in morphologic leukemia-free state (MLFS), or in partial remission (PR) after receiving the prior combination treatment.

The first time point can be, for example, prior or immediately prior to the combination treatment. The at least one of the one or more additional time points can be, for example, at the end of or after at least a cycle of the combination treatment. In some embodiments, the cycle of the combination treatment is the first cycle of the combination treatment. In some embodiments, the first time point is prior or immediately prior to a first cycle of the combination treatment, and the one or more additional time points are at the end of or after a second cycle of the combination treatment. In some embodiments, the first cycle of the combination treatment is immediately prior to the second cycle of the combination treatment.

The variant allele frequency in ctDNA can be determined, for example, by total mutation count in the ctDNA in each of the first sample and one or more additional samples, and/or by the mean variant allele frequency in each of the first sample and one or more additional samples. In some embodiments, the variant allele frequency is mutant allelic frequency (MAF) for a driver mutation of leukemia. In some embodiments, the variant allele frequency is mutant allelic frequency (MAF) for one or more driver mutations of leukemia. In some embodiments, Log₂(C₁/C₀)<a MAF threshold indicates a decrease in ctDNA MAF C₀ is ctDNA MAF in the first sample and C₁ is ctDNA MAF in one of the additional samples. In some embodiments, the MAF threshold is −0.05.

The driver mutation can be, for example, a mutation in one of the 75 genes set forth in Table 3, at least one of the one or more the driver mutations is a mutation in one of the below 75 genes ABL1, ANKRD26, ASXL1, ATRX, BCOR, BCORL1, BRAF, BTK, CALR, CBL, CBLB, CBLC, CCND2, CDC25C, CDKN2A, CEBPA, CSF3R, CUX1, CXCR4, DCK, DDX41, DHX15, DNMT3A, ETNK1, ETV6, EZH2, FBXW7, FLT3, GATA1, GATA2, GNAS, HRAS, IDH1, IDH2, IKZF1, JAK2, JAK3, KDM6A, KIT, KMT2A, KRAS, LUC7L2, MAP2K1, MPL, MYC, MYD88, NF1, NOTCH1, NPM1, NRAS, PDGFRA, PHF6, PPM1D, PTEN, PTPN11, RAD21, RBBP6, RPS14, RUNX1, SETBP1, SF3B1, SH2B3, SLC29A1, SMC1A, SMC3, SRSF2, STAG2, STAT3, TET2, TP53, U2AF1, U2AF2, WT1, XPO1, and ZRSR2, and/or one or more the driver mutations are mutations in the 75 genes. In some embodiments, the driver mutation or at least one of the one or more driver mutations is in a gene selected from the group consisting of TP53, ASXL1, DNMT3A, NRAS, SRSF2, TET2, SF3B1, FLT3, FLT3 ITD, IDH2, NPM1, RUNX1, CDKN2A, KRAS, STAG2, CALR, CBL, CSF3R, DDX41, GATA2, JAK2, PHF6, and SETBP1. In some embodiments, the driver mutation or at least one of the one or more driver mutations is in a gene selected from the group consisting of DNMT3A, TET2, NPM1, SRSF2, NRAS, CDKN2A, SF3B1, FLT3, ASXL1, SRSF2, IDH2, NRAS, and SF3B1.

In some embodiments, the method further comprises determining variant allele frequency in one or more of the ctDNA, PBMCs and BMMCs of the subject. The variant allele frequency in ctDNA can be detected, for example, using polymerase chain reaction (PCR) or next generation sequencing (NGS). In some embodiments, the variant allele frequency in ctDNA is detected using droplet digital PCR (ddPCR).

At least one of the first sample, the one or more additional samples, and the second sample can be derived from whole blood of the subject, plasma of the subject, serum of the subject, or a combination thereof. In some embodiments, the ctDNA is from whole blood of the subject, plasma of the subject, serum of the subject, or a combination thereof.

In some embodiments, the subject whose ctDNA is analyzed is undergoing or will be undergoing treatment for the leukemia or lymphoma. The method can comprise analyzing ctDNA of the subject before the treatment. The treatment can comprise one or more cycles, and the ctDNA is analyzed before, during and after one or more cycles of the treatment. For example, the ctDNA can be analyzed before, during and after two or more cycle of the treatment, three or more cycle of the treatment, or each cycle of the treatment. Each cycle of treatment can be at least 21 days, for example, 21 days, 22 days, 23 days, 24 days, 25 days, 26 days, 27 days, 28 days, 29 days, 30 days, or more, or a range between any two of these values. In some embodiments, each cycle of treatment is from about 21 days to about 28 days. In some embodiments, each cycle of treatment is from 21 days to 28 days. In some embodiments, the subject is human.

### Kits

Disclosed herein include kits, for example, for treating leukemia or lymphoma. In some embodiments, a kit comprises: a Polo-like kinase 1 (PLK1) inhibitor; and a manual providing instructions for co-administrating the PLK1 inhibitor with a B-cell lymphoma 2 (BCL-2) inhibitor to a subject for treating leukemia and lymphoma. In some embodiments, the kit comprises the BCL-2 inhibitor.

In some embodiments, the subject has leukemia. In some embodiments, the instructions comprise instructions for co-administrating the PLK inhibitor and the BCL-2 simultaneously. In some embodiments, the instructions comprise instructions for co-administrating the PLK inhibitor and the BCL-2 sequentially. In some embodiments, the instructions comprise instructions for administering of the PLK1 inhibitor orally. In some embodiments, the instructions comprise instructions for administrating the BCL-2 inhibitor orally.

In some embodiments, the instructions comprise instructions the subject has received a prior BCL-2 inhibitor treatment. In some embodiments, the instructions comprise instructions the subject did not respond to treatment with the BCL-2 inhibitor alone. In some embodiments, the instructions comprise instructions the subject is known to be resistant to a BCL-2 inhibitor therapy.

In some embodiments, the instructions comprise instructions the subject has received at least one prior treatment for leukemia or lymphoma. In some embodiments, the prior treatment does not comprise the use of a BCL-2 inhibitor, a PLK inhibitor, or both. In some embodiments, the instructions comprise instructions the subject was in remission for leukemia or lymphoma. In some embodiments, the subject in remission for leukemia was in complete remission (CR), in CR with incomplete hematologic recovery (CRi), in morphologic leukemia-free state (MLFS), or in partial remission (PR).

In some embodiments, the instructions comprise instructions for administering each of the BCL-2 inhibitor and the PLK1 inhibitor to the subject in a cycle of at least twice within a week. In some embodiments, the instructions comprise instructions for administering each of the BCL-2 inhibitor and the PLK1 inhibitor to the subject in a cycle of at least five times within a week In some embodiments, the instructions comprise instructions for administering the BCL-2 inhibitor, the PLK1 inhibitor, or both are in a cycle of at least 7 days. In some embodiments, each cycle of treatment is at least about 21 days. In some embodiments, each cycle of treatment is from about 21 days to about 28 days. In some embodiments, the instructions comprise instructions for administering the PLK1 inhibitor on at least four days in the cycle. In some embodiments, the instructions comprise instructions for not administering the PLK1 inhibitor on at least one day in the cycle. In some embodiments, the instructions comprise instructions for administrating the BCL-2 inhibitor daily. In some embodiments, the instructions comprise instructions for administrating the BCL-2 inhibitor and the PLK1 inhibitor for at least two cycles.

In some embodiments, the BCL-2 inhibitor is selective and/or specific for BCL-2 inhibition. In some embodiments, the BCL-2 inhibitor is venetoclax, obatoclax, HA14-1, navitoclax, ABT-737, TW-37, AT101, sabutoclax or gambogic acid. In some embodiments, the BCL-2 inhibitor is venetoclax.

In some embodiments, the PLK1 inhibitor is selective and/or specific for PLK1. In some embodiments, the PLK1 inhibitor is a dihydropteridinone, a pyridopyrimidine, a aminopyrimidine, a substituted thiazolidinone, a pteridine derivative, a dihydroimidazo[1,5-f]pteridine, a metasubstituted thiazolidinone, a benzyl styryl sulfone analogue, a stilbene derivative, or any combination thereof. In some embodiments, the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280. In some embodiments, the PLK1 inhibitor is onvansertib. In some embodiments, the BCL-2 inhibitor is venetoclax and the PLK1 inhibitor is onvansertib.

In some embodiments, the instructions comprise instructions for administering the PLK1 inhibitor at 12 mg/m²-90 mg/m². In some embodiments, the instructions comprise instructions for administering the BCL-2 inhibitor at 20 mg-1200 mg.

### EXAMPLES

Some aspects of the embodiments discussed above are disclosed in further detail in the following example, which are not in any way intended to limit the scope of the present disclosure.

### Example 1

### Evaluation of Onvansertib Alone and in Combination with Venetoclax in an OCI-AML3 Xenograft Model

In this example, the efficacy of onvansertib alone and in combination with venetoclax was evaluated in an OCI-AML3 xenograft model.

The OCI-AML3 xenograft model of AML is a standard xenograft model of AML that displays typical phenotypic features of NPMc+ AML, including expression of macrophage markers and lack of CD34, along with cytoplasmic expression of NPM. The OCI-AML3 cell line easily engrafts in NOD/SCID mice, maintaining the typical features of NPMc+ AML, including NPM cytoplasmic expression.

### Materials and Methods

### Test Articles

Onvansertib was provided by Trovagene (NMS-12869374 Tartarate Salt API; Lot N0900134). Venetoclax was purchased through BOC Sciences (ABT-199 CAS 1257044-40-8; Lot B19ZJ04164).

### OCI-AML3 Cells

OCI-AML3 cells (DSMZ cat no: ACC 582) were cultured in RPMI supplemented with 10% FBS and 1%P/S, in a humidified incubator at 37° C. and 5% CO₂. Normocin (Invivogen) was also added according to the manufacturer's recommendation to prevent mycoplasma growth.

### Animals

CB17 NOD.SCID mice (female, 5 weeks old) were procured through Envigo (Strain NOD.CB17-Prkdc*scidNCrHsd). Mice were fed Teklad irradiated (sterilized) mouse diet and bedded with Teklad irradiated (sterilized) corncob bedding from Envigo (Indianapolis, Ind.). Mice were housed in Optimice carousel sterile quarters with filtered air supply in disposable cages from Animal Care Systems, Inc. (Centennial, Colo.).

### OCI-AML3 Implantation

On the day of implantation (Oct. 8, 2019), OCI-AML3 cells were collected and spun at 400 ×g. Media was aspirated and cells were resuspended in 50:50 *Cultrex:PBS (w/o calcium and magnesium) at a concentration of 1×10⁸ cells/mL. A volume of 100 μL was injected into the right hind flank of each animal (a total of 1×10⁷ cells). Filled syringes and cells were kept on ice to prevent gelling of matrigel. *Cultrex: BME, type 3, 17.24 mg/mL R&D systems Cat. #3632-005-02, Lot #40498J17

### Study Arms and Treatments

Tumor volumes were monitored, and, when mean tumor volume reached ˜200 mm³, mice were stratified and placed into (4) treatment groups of (10) mice as outlined in Table 1.

TABLE 1
Dosing and schedules for current study arms.

| Group | n | Test article | Venetoclax | Onvansertib |
|---|---|---|---|---|
| 1 | 4 | 1) Vehicle | | |
| | | (Veh-1 QDx14) | | |
| | | (Veh-2 [QDx5; | | |
| | | 2-off\|x2) | | |
| 2 | 4 | 2) Venetoclax | 75 mg/kg | |
| | | (Veh-1) | | |
| 3 | 4 | 3) Onvansertib | | 30 mg/kg |
| | | (Veh-2) | | |
| 4 | 4 | 4) Venetoclax | 75 mg/kg | 30 mg/kg |
| | | (Veh-1); | | |
| | | Onvansertib | | |
| | | (Veh-2) | | |

*Vehicle #1: 10% EtOH, 30% PEG-400, and 60% phosal 50
*Vehicle #2: 0.5% w/v methyl cellulose + 0.1% v/v tween 80

Treatments were administered by oral gavage (polypropylene needles, Instech, Cat. FTP-20-38) daily for venetoclax for 14 days, and 5 days on 2 days off for two cycles for Onvansertib. For clarity, the combination was dosed so that venetoclax was given daily and the onvansertib was given 5 days on, 2 days off, just like the single agent groups.

### Results and Discussion

The OCI-AML3 model and study was conducted as described in the materials and methods section to look at efficacy of single agent venetoclax, single agent onvansertib, and the combination of both.

### Animal Health

All dosing was performed without any unexpected problems noted. Unlike the previous study, all the animals in this study had to be taken down due to tumor burden (with the exception of 1 mouse that was followed for 60 days and had a stable tumor). Therefore, the single agents and combination were tolerated at the tested dose and schedule.

The group average change in weight relative to day 0 of the study is shown in

Table 2.

TABLE 2
Relative weight change (average) for the treatment arms.

| | Day | | | | | |
|---|---|---|---|---|---|---|
| % Weight change vs Day 1 | 0 | 4 | 7 | 11 | 14 | 18 |
| Combined Vehicle | 1.00 | 1.04 | 1.09 | 1.11 | 1.15 | 1.19 |
| Venetoclax 75 mg/kg | 1.00 | 1.03 | 1.07 | 1.09 | 1.12 | 1.15 |
| Onvansertib 30 mg/kg | 1.00 | 1.04 | 1.07 | 1.10 | 1.13 | 1.18 |
| Combination | 1.00 | 1.01 | 1.03 | 1.03 | 1.05 | 1.09 |

After day 18, several animals were taken down due to excess tumor burden, and therefore the average relative weight after this point is of little use. Overall, the mice in all groups gained weight, but the combination group gained the least amount of weight. These data combined with previous data, suggest that there might still be some combined toxicity when venetoclax and onvansertib are used in combination.

### Tumor Volumes

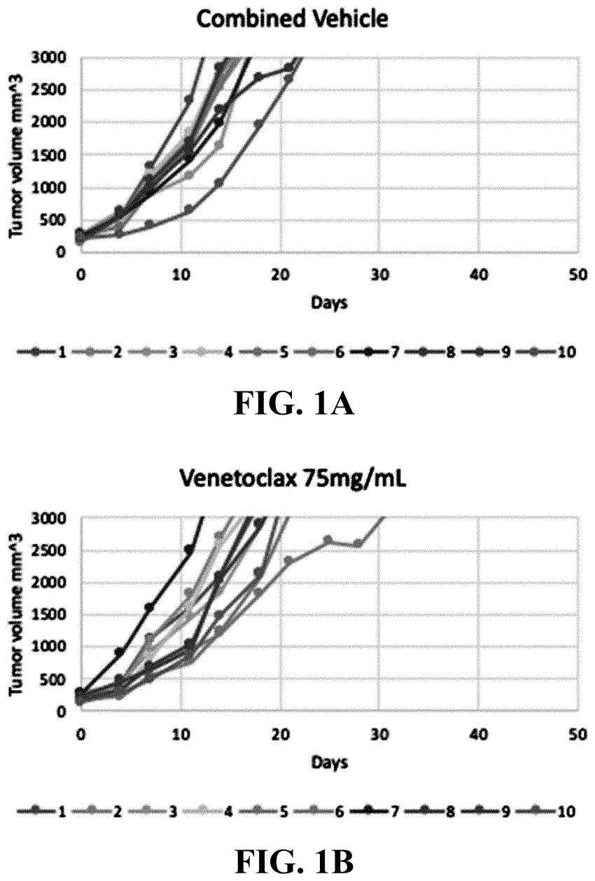

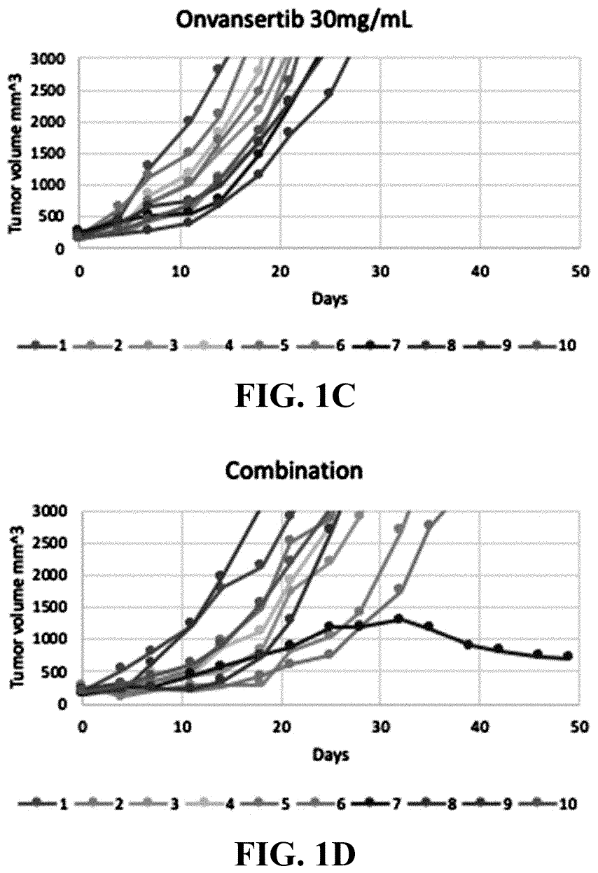

Individual tumor volumes were measured by electronic caliper 2× a week. Individual tumor volumes for each group are shown in FIGS. 1A-1D.

These data show that tumor volumes for the control mice increased quickly and no animals were left on study after 25 days. In contrast, the combination group had a less steep curve and 7 mice made it past day 25.

Comparing the average tumor volumes out to day 18, it is observed that tumor volume in the combination group was cut to less than half of the vehicle control (Table 3).

TABLE 3
Average tumor volumes through 18 days.

| | Days | | | | | |
|---|---|---|---|---|---|---|
| Average | 0 | 4 | 7 | 11 | 14 | 18 |
| Combined | 210.89 | 526.42 | 999.93 | 1567.79 | 2421.86 | 3346.72 |
| Vehicle | | | | | | |
| Venetoclax | 203.78 | 441.01 | 861.05 | 1362.15 | 2110.23 | 2856.33 |
| 75 mg/kg | | | | | | |
| Onvansertib | 208.48 | 367.54 | 708.38 | 980.01 | 1461.86 | 2269.05 |
| 30 mg/kg | | | | | | |
| Combination | 202.76 | 268.21 | 403.04 | 570.43 | 853.11 | 1246.67 |

As expected, the venetoclax group did not have tumor growth inhibition as a single agent. The onvansertib group showed some tumor growth inhibition at the 30 mg/kg level. In the previous study onvansertib had single agent activity at 40 mg/kg.

One mouse in the combination group had a stable tumor that was not growing by day 49 when the original study was set to expire. The mouse was followed out to day 60, and the tumor continued to slowly shrink out to day 60.

Using tumor volumes from day 14 (the last day with all mice on study), an ordinary ANOVA test with a Dunnett's multiple comparison was performed using Prism (graphpad v. 8.3.0) (Table 4).

TABLE 4
Dunnett's multiple comparisons test

| | Mean | Signif- | Adjusted |
|---|---|---|---|
| | Diff. | icant? | P Value |
| Vehicle vs. Venetoclax | 311.6 | No | 0.6428 |
| Vehicle vs. Onvansertib | 960 | Yes | 0.0127 |
| Vehicle vs. Combination | 1569 | Yes | <0.0001 |

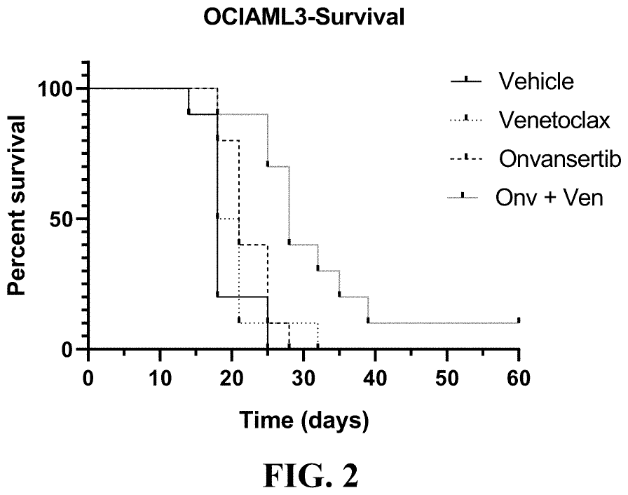

A survival graph was plotted (FIG. 2) and survival analysis (death=takedown for tumor burden limit) was also performed using Prism software (individual comparisons of survival curves using the Mantel-Cox test and Gehan-Breslow-Wilcoxon test shown in Table 5).

TABLE 5
Statistical analysis of survival

| | Comparison of Survival Curves | | |
|---|---|---|---|
| | Vehicle vs. | | |
| | Venetoclax | Onvansertib | Combination |
| Log-rank (Mantel-Cox) test | | | |
| Chi square | 0.4687 | 3.577 | 13.17 |
| df | 1 | 1 | 1 |
| P value | 0.4936 | 0.0586 | 0.0003 |
| P value summary | ns | ns | *** |
| Are the survival curves sig | No | No | Yes |
| different? | | | |
| Gehan-Breslow-Wilcoxon test | | | |
| Chi square | 0.7625 | 5.221 | 12.24 |
| df | 1 | 1 | 1 |
| P value | 0.3825 | 0.0223 | 0.0005 |
| P value summary | ns | * | *** |
| Are the survival curves sig | No | Yes | Yes |
| different? | | | |

*Gehan-Breslow-Wilcoxon uses higher weighing for early events.

### Conclusions

The OCI-AML3 xenograft is a human acute myeloid leukemia model that has been shown to be resistant to venetoclax therapy and is used in testing combinations that might improve sensitivity. In the present study, single agent venetoclax (75 mg/kg), single agent onvansertib (30 mg/kg), and the combination were tested for efficacy. Activity was noted for single agent onvansertib, but not for single agent venetoclax. The combination group exhibited synergistic activity, with one mouse surviving to 60 days past the start of treatment

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of treating leukemia or lymphoma, the method comprising: administrating a B-cell lymphoma 2 (BCL-2) inhibitor and a Polo-like kinase 1 (PLK1) inhibitor to a subject with leukemia or lymphoma, thereby inhibiting progression of the leukemia or lymphoma.

2. The method of claim 1, wherein the subject has leukemia or lymphoma.

3. The method of claim 2, wherein the leukemia is acute myeloid leukemia (AML), myelodysplastic syndrome (MDS), acute lymphocytic leukemia, or chronic myelomonocytic leukemia (CMML), and/or wherein the lymphoma is a Hodgkin lymphoma or a Non-Hodgkin lymphoma.

4. (canceled)

5. (canceled)

6. (canceled)

7. (canceled)

8. The method of claim 1, wherein the leukemia or the lymphoma is advanced, metastatic, refractory, and/or relapsed.

9. The method of claim 1, wherein the PLK inhibitor and the BCL-2 are co-administered simultaneously or administered sequentially.

10. (canceled)

11. The method of claim 1, wherein the administration of the PLK1 inhibitor, the BCL-2 inhibitor, or both is oral administration.

12. (canceled)

13. The method of claim 1, wherein the inhibition of the leukemia or lymphoma progression is greater than the combined inhibition of progression caused by the BCL-2 inhibitor alone plus the PLK1 inhibitor alone.

14. The method of claim 1, wherein the subject achieves a complete response.

15. (canceled)

16. (canceled)

17. (canceled)

18. The method of claim 1, wherein the BCL-2 inhibitor and the PLK1 inhibitor are each administered to the subject in a cycle of at least twice within a week.

19. (canceled)

20. The method of claim 1, wherein the BCL-2 inhibitor, the PLK1 inhibitor, or both are administered in a cycle of at least 7 days.

21. (canceled)

22. (canceled)

23. The method of claim 20, wherein the PLK1 inhibitor is administered on at least four days in the cycle.

24. (canceled)

25. The method of claim 1, wherein the BCL-2 inhibitor is administered daily.

26. (canceled)

27. (canceled)

28. The method of claim 1, wherein the BCL-2 inhibitor is venetoclax, obatoclax, HA14-1, navitoclax, ABT-737, TW-37, AT101, sabutoclax or gambogic acid.

29. (canceled)

30. (canceled)

31. (canceled)

32. The method of claim 1, wherein the PLK1 inhibitor is onvansertib, BI2536, Volasertib (BI 6727), GSK461364, AZD1775, CYC140, HMN-176, HMN-214, rigosertib (ON-01910), MLN0905, TKM-080301, TAK-960 or Ro3280.

33. The method of claim 1, wherein the PLK1 inhibitor is onvansertib.

34. The method of claim 33, wherein onvansertib is administered at 12 mg/m²-90 mg/m².

35. The method of claim 33, wherein the concentration of onvansertib in a blood of the subject satisfies at least one of the criteria:
(i) a maximum concentration (Cₘₐₓ) of onvansertib in the blood of the subject is from about 100 nmol/L to about 1500 nmol/L,
(ii) an area under curve (AUC) of a plot of the concentration of onvanserib in the blood of the subject over time is from about 1000 nmol/L.hour to about 400000 nmol/L.hour,
(iii) a time (Tₘₐₓ) to reach the maximum concentration of onvansertib in the blood of the subject is from about 1 hour to about 5 hours, and/or
(iv) an elimination half-life (T_{1/2}) of onvansertib in the blood of the subject is from about 10 hours to about 60 hours.

36. (canceled)

37. (canceled)

38. (canceled)

39. (canceled)

40. (canceled)

41. (canceled)

42. (canceled)

43. (canceled)

44. The method of claim 1, further comprising determining leukemia or lymphoma status of the subject and/or responsiveness of the subject to a PLK1 inhibitor treatment.

45. (canceled)

46. (canceled)

47. (canceled)

48. A kit comprising: a Polo-like kinase 1 (PLK1) inhibitor; and a manual providing instructions for co-administrating the PLK1 inhibitor with a B-cell lymphoma 2 (BCL-2) inhibitor to a subject for treating leukemia and lymphoma.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. The kit of claim 48, further comprising the BCL-2 inhibitor.
